(19) United States
(12) Patent Application Publication
(10) Pub. No.: US 2023/0298755 A1
(43) Pub. Date: Sep. 21, 2023

(54) Data labelling method for building a database for configuring, validating and/or testing an application for monitoring an individual's fatigue level

(71) Applicant: THALES, COURBEVOIE (FR)

(72) Inventor: Bastien BERTHELOT, TOULOUSE (FR)

(21) Appl. No.: 18/183,938

(22) Filed: Mar. 14, 2023

(30) Foreign Application Priority Data
Mar. 15, 2022 (FR) 2202257

(51) Int. Cl. G16H 50/20; B64D 11/00

(52) U.S. Cl. CPC G16H 50/20; B64D 11/00

(57) ABSTRACT

-- A method including acquisition of useful data during an experiment phase, the useful data being physiological data obtained by means of sensor(s) and each corresponding to an input data of the monitoring application, acquisition of declarative data of the individual, in real-time during the experimental phase and/or in differed time before and/or after the experimental phase, merging of useful data and declarative data in order to calculate a true level of fatigue, labeling the useful data with the calculated true level of fatigue, and storing the labeled useful data in the learning database.

### REFERENCE TO RELATED APPLICATIONS

This application claims priority of French Patent Application No. 22 102257, filed on Mar. 15, 2022.

### TECHNICAL FIELD OF THE INVENTION

The general field of the present invention is that of means of evaluating, in real-time, a level of fatigue of an individual, e.g. the level of fatigue of a pilot of an aircraft, or the level of fatigue of an air traffic controller working in front of a radar display console, or further the level of fatigue of a driver of a motor or rail vehicle.

### BACKGROUND OF THE INVENTION

Indeed, when the level of fatigue, e.g. of an aircraft pilot, becomes too significant, the pilot’s vigilance is strongly reduced to the point of no longer being able to perform the tasks required for continuing a safe flight.

Since the pilot is generally not aware of a state such as drowsiness, and thus cannot detect such a state, it is useful to implement automatic monitoring procedures for evaluating the instantaneous level of fatigue of the pilot and for initiating a countermeasure when it is detected that the level of fatigue is at a critical level.

In particular, physiological monitoring applications are known, which implement algorithms for analyzing certain relevant physiological variables of the pilot under surveillance, collected by means of various sensors. We can cite the example of an electroencephalogram measuring the brain activity of the pilot for evaluating the level of fatigue of the pilot in real time.

Such monitoring applications are calibrated and/or validated and/or tested using labeled datasets, i.e. datasets for which the true level of fatigue that the data describe is known (notion of “ground truth”). It is then a question of using the labeled datasets for adjusting the monitoring application parameters so that the level of fatigue evaluated by using the monitoring application once the parameters thereof are correctly set, corresponds to the true level of fatigue.

It appears thereby that the labeling of a dataset is a crucial step for the monitoring method to be configured in such a way as to be efficient.

Currently, data labeling is done by experts. Same requires a long analysis period. Hence, labeling is expensive.

Moreover, since labeled datasets result from a method carried out by experts, the labeled datasets obtained are few in number, have a small number of data and a reduced variability. Hence, labeled datasets do not support the robust training of the monitoring methods.

There is thus a need to automate the labeling of data, which will allow labeling to be extended to processing data with a greater number of components in order to better describe the level of fatigue of the monitored individual, and to qualify more data so as to cover more situations. The resulting labeled datasets will allow monitoring applications to be trained more robustly in order to more accurately estimate the instantaneous level of fatigue of the monitored individual.

The article by LL JUE et al, “Identification and classification of construction equipment operator’s mental fatigue using wearable eye-tracking technology”, AUTOMATION IN CONSTRUCTION, ELSEVIER, AMSTERDAM, NL, vol. 109, Oct. 31, 2019, D1 automatically evaluates the mental fatigue of an operator in a non-invasive way using eye movement tracking. According to the above document, a first step of collecting useful data, a second step of automatic identification of a plurality of levels of fatigue, a third step of automatic labeling of useful data with a level of fatigue, and a fourth step of training a classification algorithm, the inputs of which correspond to the useful data.

### SUMMARY OF THE INVENTION

The subject matter of the present invention is thus an alternative data labeling method for affixing a label such as the level of fatigue on data collected in real-life situations, during a training flight or during a simulation session.

To this end, the invention relates to a data labeling method and a computer program product for implementing the above labeling method.

### BRIEF DESCRIPTION OF THE DRAWING

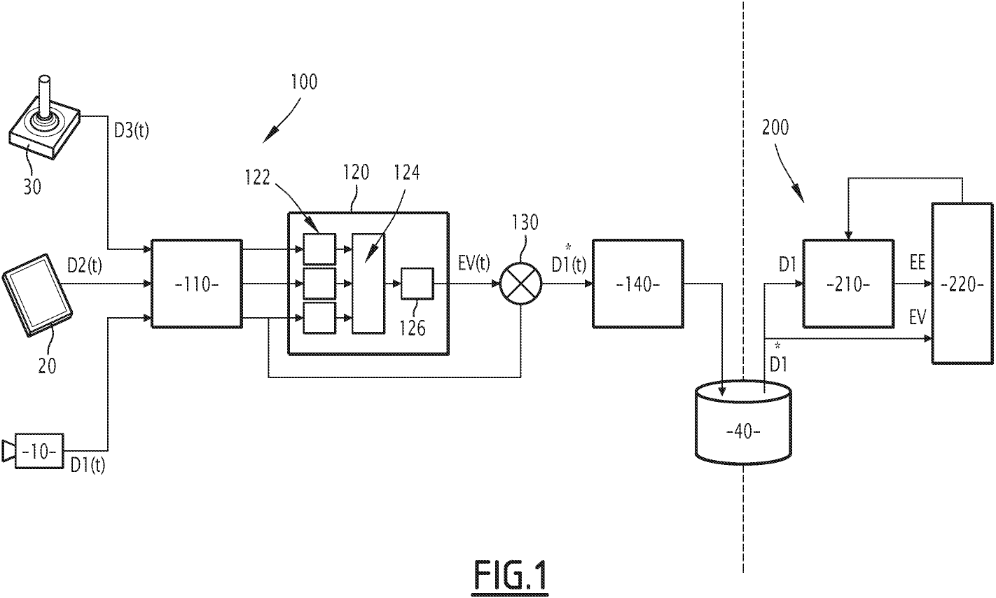

The invention and the advantages thereof will be better understood on reading the detailed description which follows, of a particular embodiment, given only as an example, but not limited to, the description being made with reference to the single enclosed Figure [FIG. 1] which shows, in the form of blocks, a preferred embodiment of the labeling method according to the invention.

### DETAILED DESCRIPTION

A preferred embodiment of the labeling method according to the invention will be presented with reference to the enclosed figure.

Preferentially, the labeling method according to the invention is implemented in the form of software executed by a computer. The computer includes computing resources, such as a processor, means of storage, such as a memory, and interface means, such as input/output ports for connection to one or a plurality of sensors, one or a plurality of human-machine interfaces, or one or a plurality of systems from which the computer receives data to be merged. The means of storage store the instructions of computer programs, in particular a program, the execution of which implements the method according to the invention.

In the figure, the labeling method 100 automatically fills a learning database 40 from the labeled data, from which a configuration method 200 of a monitoring application of the level of fatigue of a pilot can be implemented.

### Monitoring Application

In the present embodiment, the monitoring application, once properly configured, is used for real-time monitoring of the level of fatigue of a pilot in a control station of an aircraft, whether the control station is e.g. on-board the aircraft or remote in the case of a drone.

The monitoring application relies on a first set of physiological data, called useful data, as input data to the application for estimating the instantaneous level of fatigue of the pilot.

The useful data are acquired by one or a plurality of sensors provided either on the pilot as such (in the case of a carried sensor) or in the aircraft control station (remote sensor).

Each sensor is configured for measuring at least one instantaneous physiological variable of the pilot, which is related to the level of fatigue of the pilot.

If appropriate, the raw data delivered by a sensor undergoes preprocessing in order to generate useful data which can be applied as input to the monitoring application.

The real-time processing of the acquired useful data allows the monitoring application to estimate an instantaneous level of fatigue of the pilot and advantageously to automatically trigger a suitable response whenever the level of fatigue is deemed incompatible with the pilot’s mission.

The monitoring application e.g. is suitable for classifying the state of the pilot, as characterized by the useful data either in a “nominal fatigue” class or in a “dangerous fatigue” class. In a variant or in combination, the monitoring application is apt to quantify the level of fatigue of the state of the pilot with a continuous scale, e.g. graduated over a hundred points.

### General Information

The labeling method 100 is based on the processing of data of different modalities (or natures) for the determination of a true (or ground) instantaneous level of fatigue.

The labeling method 100 includes a first data acquisition step 110, a second data merging step 120, a third data labeling step 130 with a true level of fatigue, and a fourth step 140 of storing the data thus labeled in the learning database 40.

### Step 110

The data acquired during the step 110 are of two types: first, there is the useful data forming a first set of data and which form the inputs to the monitoring application; then there is the labeling data forming a second set of data and which are not inputs to the monitoring application, but which are used in the labeling method to better describe the state of the pilot and are used for determining the true level of fatigue more accurately.

The first step 110 is implemented during an experimental flight so as to reproduce as well as possible the actual conditions in which the monitoring application will be implemented.

The first step 110 is aimed at collecting data from the different modalities:

First, it is a question of collecting raw useful data D1. On the figure, the means of acquiring the first set of data are generally indicated by the number 10. It is a question of using sensors which are identical or similar to the sensors used when implementing the monitoring application.

The sensors used are preferentially minimally or not very invasive, for continuously measuring one or a plurality of relevant physiological variables. It is a question e.g. of implementing a camera for observing the pilot’s face in order to obtain, after a suitable preprocessing, preferentially carried out a posteriori, an instantaneous eye activity or even a movement of the pilot in response to a given stimulus (operational situation, information displayed on a human/machine interface). It is a question e.g. of implementing a sensor, such as a connected watch, for measuring the instantaneous heart rate of the pilot. It is a question e.g. of implementing a sensor integrated into an earplug, for measuring the temperature and the oxygenation rate of the pilot’s blood.

The next step is to collect raw labeling data D2. On the figure, the means of acquiring the second set of data are generally indicated by the number 20.

Labeling data are physiological variables which are very strongly correlated with the neuropsychological state of the pilot. However, the measurement of such variables involves sensors which cannot be implemented in a real-life case, because same are too invasive and would then be difficult to accept by the pilot in flight, or not very robust and would not withstand being used in the highly variable conditions of aeronautical operations.

In particular, the sensors used in the acquisition of labeling data are selected from the group consisting of: a cardiac sensor, in particular an electrocardiograph; a pulse oximeter, in particular a photoplethysmography sensor; a respiration sensor; an accelerometer; a scalp electrode, e.g. an electroencephalograph; a pressure sensor arranged in the operator’s seat; a pressure sensor arranged in a control device suitable for being actuated by the operator; a sweating sensor for the operator; a galvanic skin response sensor; a camera configured for taking at least one image comprising at least part of the operator, in particular the eyes for eye tracking; a microphone; an infrared sensor of the temperature of the operator’s skin; an internal temperature sensor for the operator; and a near-infrared spectroscopy headband (also called “fNIRS” headband) using near-infrared light for monitoring brain activity.

The labeling data advantageously further include declarative data, obtained by means of one or a plurality of suitable interfaces for interrogating the pilot on his/her feeling, in real-time during the experimental flight and/or in differed time before and/or after the experimental flight. The above are discrete subjective declarative data acquired at low frequency so as not to hinder the pilot during the flight. Before the flight, the declarative data entered by the pilot can e.g. be used for defining a general category to which the pilot belongs or for defining the operational scenario that the experimental flight will allow to be tested. Examples include the Karolinska Sleepiness Scale (KSS), the seven-tiered Samn-Perelli fatigue scale (SPS), a continuous visual fatigue scale, or sleep-specific elements (waking hours, sleeping hours over the last three nights, etc.)

Advantageously, it is also a question of collecting environmental data D3. On the figure, the means of acquiring the third set of data are generally indicated by the number 30. The data relate to the environment in which the flight takes place, the actions of the pilot and the pilot’s performance in the management of the systems with which the aircraft is equipped. It is a question e.g. of recording the control actions performed by the pilot on the piloting systems of the aircraft, or the response times to the execution of a task following the occurrence of a particular event during the flight. It is also a question e.g. of recording errors or alarm failures. It is a question of continuous acquisition. Even if some recordings are discrete, the corresponding acquisition means are permanently on standby.

The raw data acquired by each of the devices 10, 20 and 30 are dated with the instant of acquisition t thereof. The data D1(t), D2(t) and D3(t) are preferentially recorded in the learning database 40 at each instant of the experimental flight.

### Step 120

The labeling method 100 continues with a second step 120 of merging the data acquired during step 110 and relating to the same experimental flight.

The second step is preferentially performed off-line, i.e. after the flight.

The raw data stored in the training database 40 are analyzed during such step in order to build a labeled dataset.

Preferentially, during a first elementary step 122, each type of raw data undergoes a suitable preprocessing. In particular, the raw useful data are preprocessed so as to obtain useful data, for which the format corresponds to the data applied as input to the monitoring application. E.g. the images acquired by the camera are analyzed so as to extract instantaneous eye movements of the pilot.

Moreover, during the step 122, the data are synchronized in the sense that a time step is defined and the data are dated with respect to the new time sampling. If a datum has a sampling frequency higher than the time step, a mean value of the value of the datum is taken into account. If a datum has a sampling frequency lower than the time step, the value of the datum is repeated at each time step, between two updates of the datum.

For each time step, a first data vector including the useful data D1 and a second data vector including the labeling data D2 and advantageously the environment data D3 are thus obtained,.

The use of data from the different modalities D2 and D3 provide an overall description of the state of the pilot and of the environment.

The step 120 continues with an elementary analysis step 124 used for determining, for each second data vector, the best possible value of the level of fatigue as the true level of fatigue EV for the time step considered. Hence, the above is an instantaneous variable. Same is a continuous information obtained over the entire experimental flight.

The possible values of the true level of fatigue EV are the values of the output of the monitoring application to be configured. The possible values e.g. belong to the set comprising at least two categories: a normal level of fatigue and a dangerous level of fatigue. In a variant, the set of possible values has more than two classes. In addition to the previous two levels e.g., the set includes a level of drowsiness, a level of sleep and a level of fainting. Further e.g., the classification is based on three levels of fatigue, low, moderate and high, respectively.

In another variant, which can advantageously be implemented in parallel with the preceding variants, the possible value of the true level of fatigue EV is a continuous variable, e.g. between 0 and 100, corresponding e.g. to the probability that the state of the pilot corresponds to the dangerous level of fatigue.

For the elementary analysis step 124, a supervised learning algorithm is advantageously executed for determining the possible values of the true level of fatigue from the second data vectors (and, if appropriate, the third data vectors) collected during the entire training flight.

Such a supervised learning algorithm is e.g. a neural network, an SVM (Support Vector Machines), a KNN (k-nearest neighbors), a logistic regression, etc. The labeling data and D and, if appropriate, the environment data D3, thus form the input data to the supervised learning algorithm. In the field of artificial intelligence, in English, the input data are commonly called “features”.

In a variant, the algorithm executed is: an unsupervised learning algorithm, such as hierarchical grouping, k-means, Gaussian mixtures, self-organizing map, etc.; or an optimal filtering algorithm, such as the Kalman filtering and/or particle filtering; or an empirical unbiased modeling algorithm, such as a decision tree, etc.

Advantageously, the step 120 comprises an elementary step 126 of filtering the level of fatigue true EV. It is a question e.g. of comparing the level of fatigue at the current instant t with the levels of fatigue at the previous instants in order to avoid too rapid and hence erroneous evolutions of such variable. The level of fatigue e.g. at the current instant t is weighted by a mean value of the levels of fatigue at the previous instants over a time window of predefined width.

At the output of step 120, for each time step, a true level of fatigue EV is obtained.

The labeling dynamics, i.e. the time step chosen for the merging step, can be adapted depending on the monitoring application. For the detection e.g. of a level of fatigue corresponding to drowsiness, which is a slow process, the dynamic of labeling can be slower, by choosing a longer time step. If e.g. a value of level of fatigue is wanted every second, the sampling step during the step 120 is 1 Hz.

### Step 130

In order to label the useful data at time t during the step 130, i.e. the first data vector at an instant t, a time correlation is established with the labeling data at the instant t, i.e. the second data vector at the instant t, and the value of the true level of fatigue EV calculated for the second data vector at the instant t during the step 120 is associated with the useful data D1 at the instant t so as to form useful data labeled D*1 at the instant t, i.e. a first vector of data labeled at the instant t.

### Step 140

Finally, during the step 140, the useful data labeled D*1 at the instant t are recorded in the learning database 40.

### Configuration Method 200

The configuration method 200 consists of training the monitoring application.

During the step 210, the monitoring application, configured with a first set of configuration parameters, is executed on each labeled data vector of a set of vectors extracted from the base 40, so as to obtain an estimated level of fatigue EE of the vector.

During the step 220, the estimated level of fatigue EE is compared with the value of the true level of fatigue EV for each vector in the vector set used during the step 210. Such comparison step leads to an update of the configuration parameters of the monitoring application.

The iteration of steps 210 and 220 over all or part of the useful data labeled D*1 of the database 40 is used for optimizing the configuration parameters of the monitoring application.

Such iterative method is stopped when a convergence criterion of the configuration parameters is verified, or, easier, after a certain number of iterations of the iterative process. The monitoring application is then considered as having been configured.

In a variant, instead of being completely disjoint, a part of the second set of labeling data overlaps with the first set of useful data.

### Benefits

The labeling method according to the invention can be used for labeling data acquired during a flight by meeting the following criteria:
the method can be used for automatically collecting data in an environment similar or identical to the environment where the target monitoring application will be implemented;
the method can be used for finely determining the true level of fatigue at an instant t, to be associated with the useful data, yet using labeling data different from the useful data. Thereby, the monitoring application does not need to input the labeling data and is limited to taking into account only a small number of useful data. The implementation of the monitoring application in a real-life case does not require equipping the cockpit and/or the pilot with complex sensors. The above thus disrupts less the pilot’s activities during the implementation phase of the monitoring application;
in this way, data can be collected continuously over the entire experiment, unlike questionnaires and attention tests;
the present method has greater objectivity than the labeling methods based solely on pilot declarations.

The database built by implementing the present labeling method can be used not for training the target application, but for validating or testing same.

1. A method for labeling useful data to build a database for configuring, validating and/or testing an application for monitoring the level of fatigue of an individual, the method comprising, for each of a plurality of time steps:
acquiring useful data during an experimental phase, the useful data comprising physiological data obtained by one or a plurality of sensors and each physiological data corresponding to an input data to the monitoring application;
acquiring labeling data, in real-time during the experimental phase and/or at a different time before and/or after the experimental phase;
merging the useful data and the labeling data;
computing from the labeling data a true level of fatigue for each instant of the experimentation phase;
labeling the useful data acquired at an instant with the true level of fatigue computed for the labeling data acquired at that instant; and
storing the useful data labeled in the learning database.

2. The method according to claim 1, further comprising acquiring environment data, said merging using the environment data in addition to the labeling data, for computing the true level of fatigue for each instant of the experimentation phase.

3. The method according to claim 1, wherein the true level of fatigue is selected from at least two levels.

4. The method according to claim 1, wherein said acquiring labeling data uses a sensor selected from:
a cardiac sensor
a pulse oximeter
a respiration sensor;
an accelerometer;
a scalp electrode ;
a pressure sensor arranged in the operator’s seat;
a pressure sensor arranged in a control device suitable for being actuated by the operator;
a sweating sensor for the operator;
a galvanic skin response sensor;
a camera configured for taking at least one image comprising at least part of the operator ;
a microphone;
an infrared sensor of the temperature of the operator’s skin;
an internal temperature sensor for the operator; and
a near-infrared spectroscopy headband.

5. The method according to claim 1, wherein said merging comprises:
a supervised learning algorithm ;
an unsupervised learning algorithm, ;
an optimal filtering algorithm ; or
an empirical unbiased modeling algorithm .

6. The method according to claim 1, wherein, the acquired data are raw data, and said merging comprises preprocessing the raw data to obtain processed data.

7. The method according to claim 6, wherein said merging further comprises synchronizing the raw data according to a selected time step.

8. The method according to of claim 1, wherein said merging comprises filtering the true level of fatigue computed for each time step.

9. A computer program comprising software instructions which, when executed by a computer, cause the computer to perform a method for labeling according to claim 1.

10. The method according to claim 4, wherein the cardiac sensor comprises an electrocardiograph.

11. The method according to claim 4, wherein the pulse oximeter comprises a photoplethysmography sensor.

12. The method according to claim 4, wherein the scalp electrode comprises an electroencephalograph.

13. The method according to claim 4, wherein the at least one image comprises the eyes of the operator, for eye tracking.

14. The method according to claim 5, wherein the supervised learning algorithm comprises one or more of neural networks, support vector machines, k nearest neighbors, and logistic regression.

15. The method according to claim 5, wherein the unsupervised learning algorithm comprises one or more of hierarchical clustering, k-means, Gaussian mixtures, and a self-organizing map.

16. The method according to claim 5, wherein the optimal filtering algorithm comprises one or both of Kalman filtering and particle filtering.

17. The method according to claim 5, wherein the empirical unbiased modeling algorithm comprises a decision tree.
